(19) United States
(12) Patent Application Publication
(10) Pub. No.: US 2024/0008785 A1
(43) Pub. Date: Jan. 11, 2024

(54) INFORMATION PROCESSING SYSTEM, INFORMATION PROCESSING DEVICE, INFORMATION PROCESSING METHOD, AND INFORMATION PROCESSING PROGRAM

(71) Applicants: Michio Yokoyama, Yonezawa-shi, Yamagata (JP); Tomochika Harada, Yonezawa-shi, Yamagata (JP); Yume Cloud Japan Inc., Yonezawa-shi, Yamagata (JP)

(72) Inventors: Michio Yokoyama, Yonezawa-shi, Yamagata (JP); Tomochika Harada, Yonezawa-shi, Yamagata (JP); Daisuke Yoshida, Yonezawa-shi, Yamagata (JP); Makoto Shohara, Yonezawa-shi, Yamagata (JP); Kenichi Suzuki, Yonezawa-shi, Yamagata (JP); Shigeyuki Seko, Yonezawa-shi, Yamagata (JP)

(21) Appl. No.: 18/277,691

(22) PCT Filed: Feb. 14, 2022
(86) PCT No.: PCT/JP2022/005712

§ 371 Date: Aug. 17, 2023

(30) Foreign Application Priority Data
Feb. 17, 2021 (JP) 2021-023407

(51) Int. Cl. A61B 5/16; G10L 25/63; G10L 25/66; G06V 40/16

(52) U.S. Cl. CPC A61B 5/165; G10L 25/63; G10L 25/66; G06V 40/176

(57) ABSTRACT

An information processing device connected to a terminal device of a subject and which visualizes emotion of the subject includes a data managing section that acquires at least data related to voice, a facial expression image, and a pulse wave of the subject; an emotion expression engine section which calculates a brain fatigue level based on a frequency of the voice, which calculates a mood level by extracting an emotion of the subject from the facial expression image, and which calculates a stress level by performing a frequency analysis of the pulse wave by fast Fourier transform and extracting a high-frequency section and a low-frequency section; and a three-axes processing section which displays a graph of points plotted at coordinates corresponding to the brain fatigue level, the mood level, and the stress level in a three-dimensional space defined by an X-axis, a Y-axis, and a Z-axis.

### TECHNICAL FIELD

This disclosure relates to an information processing system, an information processing device, an information processing method, and an information processing program (“information processing system and the like”) which analyze and visualize various kinds of data such as a medical questionnaire, voice, a facial expression, a pulse wave/heart rate of a subject to contribute towards solving social problems by realizing early detection of a mental illness.

Specifically, this disclosure relates to an information processing system and the like which, by having a subject (user) use a questioning/examination site on a network such as the Internet to fill in a questionnaire (stress check) related to stress and subsequently engage in a video chat with a counselor or use a pulse wave meter, acquire questionnaire data, facial expression image data, voice data, pulse wave/heart rate data and the like of the subject, calculate values of a stress level, a brain fatigue level, and a mood level based on the various kinds of acquired data and, by plotting the calculated values in a three-dimensional space defined by an X-axis, a Y-axis, and a Z-axis, visualize the subject's emotion (mental status) and the like to support diagnosis and treatment.

### BACKGROUND

In recent years, while the popularization of information technology (IT) has led to rapid simplification and improved convenience in communication, there is an upward trend in the number of people with mental illnesses caused by a lack of communication, work-related stress, fatigue and the like. Therefore, diagnostic systems are being proposed which, by quantitatively measuring and objectively assessing data related to a stressed state or a fatigued state of a person (subject), enables the subject himself/herself to readily comprehend fatigue/stress.

For example, a fatigue/stress examination system described in Japanese Patent Laid-Open No. 2015-054002 is capable of analyzing, using a cloud-side analysis server, electrocardiogram/pulse wave data measured by an electrocardiogram monitor/pulse wave meter, comprehending a state of stress in terms of a numerical value from a balance and strength of autonomic nerves, transmitting the analyzed data to a client terminal, and visually displaying the analyzed data on the client terminal.

In addition, a health value estimation system described in Japanese Patent Laid-Open No. 2018-181004 constructs an estimation model by classifying characteristic behavior (behavioral features) that appear under stress into a plurality of clusters and converting the characteristic behavior (behavioral features) into numerical values from action history including position information and movement information acquired from various sensors included in a mobile terminal such as a smartphone, turning on/off of power, logs related to activation of applications, the number of times of telephone use and the like, and learning, by machine learning, a relationship with a stressed state based on heart rate data measured in advance. Furthermore, by collating a numerical value of a behavioral feature newly acquired using the mobile terminal such as a smartphone with the constructed estimation model, a health value indicating a state of health of the subject himself/herself can be estimated.

The fatigue/stress examination system described in JP '002 simultaneously measures an electrocardiogram and a pulse wave of a subject, measures a state of autonomic nerves of the subject from the electrocardiogram/pulse wave data, and provides unified management of fatigue/analysis result data so that a degree of fatigue and a stress tendency are visualized as numerical values. However, since the fatigue/analysis result data does not reflect subjective determination results based on voice and facial expressions of the subject himself/herself that are obtained during questioning or an interview by a doctor, an industrial physician, a health nurse or the like, there is a possibility that an analysis of emotion of the subject cannot be realized with high accuracy.

In addition, the health value estimation system described in JP '004 is capable of constructing an estimation model using accurately quantified data which can become more suitable training data in supervised machine learning and capable of estimating a health value of the subject himself/herself. However, since the health value estimation system cannot accurately quantify a depressive mood when the subject himself/herself is unaware of being melancholic, subjective determination results based on voice and facial expressions of the subject himself/herself that are obtained during questioning or an interview by a doctor, an industrial physician, a health nurse or the like cannot be used as training data. As a result, since an estimation model that sufficiently reflects an emotion (mental status) of the subject cannot be constructed, there is a possibility that the health value estimation system described in JP '004 is also unable to realize an analysis of emotion of the subject with high accuracy.

It could therefore be helpful to provide an information processing system and the like which acquire not only quantitative data (a pulse wave/heart rate and the like) of the subject measured by measuring instruments but also data of a stress check result based on a stress check questionnaire (Stress Check System Introduction Manual—Ministry of Health, Labour and Welfare (URL https://www.mhlw.go.jp/bunya/roudokijun/anzeneisei12/pdf/150709-1.pdf) (Retrieved Feb. 15, 2021)) and data such as voice, a facial expression image or the like of the subject during counseling using a communication tool such as a video chat (video call), calculate values of a stress level, a brain fatigue level, and a mood level from the data and, by plotting the calculated values in a three-dimensional space defined by an X-axis, a Y-axis, and a Z-axis, visualize the subject's emotion (mental status) and the like and enable diagnosis and treatment to be assisted.

### SUMMARY

We thus provide:

An information processing device connected to a terminal device of a subject and which visualizes emotion of the subject includes:
a data managing section which acquires at least data related to voice, a facial expression image, and a pulse wave of the subject;
an emotion expression engine section which calculates a brain fatigue level based on a frequency of the voice, which calculates a mood level by extracting an emotion of the subject from the facial expression image, and which calculates a stress level by performing a frequency analysis of the pulse wave by fast Fourier transform and extracting a high-frequency section and a low-frequency section; and
a three-axes processing section which displays a graph of points plotted at coordinates corresponding to the brain fatigue level, the mood level, and the stress level in a three-dimensional space defined by an X-axis, a Y-axis, and a Z-axis, wherein
the data related to the voice is acquired by making an audio recording of at least a part of a video call with the subject via the terminal device,
the data related to the facial expression image is acquired by making a video recording of at least a part of a video call with the subject via the terminal device, and
the data related to the pulse wave is acquired via the terminal device from a pulse wave meter that measures a pulse wave of the subject.

Preferably, the data managing section associates the data related to the voice, the facial expression image, and the pulse wave of the subject with dates and times of acquisition of the data and stores the data in storage means of the information processing device, and
the three-axes processing section displays a graph of points plotted according to a time series at coordinates corresponding to the brain fatigue level, the mood level, and the stress level of the subject for each of the dates and times in the three-dimensional space.

Preferably, the three-dimensional space is divided into a plurality of per-type classification categories, and
the three-axes processing section notifies a category to which a point of coordinates corresponding to the brain fatigue level, the mood level, and the stress level of the subject belongs among the plurality of per-type classification categories in the three-dimensional space.

Preferably, an improvement plan to be proposed to the subject is determined for each of the plurality of per-type categories, and
the emotion expression engine section notifies the improvement plan with respect to the category to which a point of coordinates corresponding to the brain fatigue level, the mood level, and the stress level of the subject belongs in the three-dimensional space.

Preferably, the data related to the voice is data acquired by making a continuous audio recording of the voice of the subject reading out loud predetermined fixed phrases displayed on the terminal device at least until a predetermined audio recording time is reached during a video call with the subject via the terminal device.

Preferably, the emotion expression engine section executes a cerebral activity index measurement algorithm for measuring CEM values that each represents a cerebral activity index to acquire one or more of the CEM values for each subject from the data related to the voice, and the brain fatigue level is an average value of the one or more CEM values.

Preferably, the data related to the pulse wave is data acquired by dividing a pulse wave measured by the pulse wave meter into sections, each section being a predetermined time interval.

Preferably, the emotion expression engine section divides, for each section of the pulse wave, the pulse wave in the section into Hamming windows and calculates, with respect to the pulse wave in each of the Hamming windows, a pulse interval PPI being an interval from a peak to a next peak of the pulse wave of one heartbeat and a time of day,
the emotion expression engine section generates, for each section of the pulse wave, a time-PPI graph which plots a point at coordinates corresponding to the pulse interval PPI and the time of day in a two-dimensional space defined by time of day as an axis of abscissa and PPI as an axis of ordinate,
the emotion expression engine section interpolates between discrete values in the time domain-PPI graph and applies a fast Fourier transform FFT, and calculates an LF value corresponding to the low-frequency component, an HF value corresponding to the high-frequency component, and an LF/HF value by respectively integrating a power spectral density PSD of a result of the FFT in the low-frequency section and in the high-frequency section, and
the stress level is based on at least one value among the LF value, the HF value, and the LF/HF value.

Preferably, the low-frequency section is 0.04 Hz or higher and lower than 0.15 Hz, and
the high-frequency section is 0.15 Hz or higher and lower than 0.4 Hz.

Preferably, the data related to the facial expression image is data acquired by making a continuous video recording of a moving image of a facial expression of the subject until at least a predetermined video recording time is reached during a video call with the subject via the terminal device.

Preferably, the emotion expression engine section executes a facial expression recognition algorithm to count each of a plurality of emotion expressions recognized from a moving image of a facial expression of the subject included in the data related to the facial expression image,
the emotion expression engine section calculates a proportion for each of the plurality of emotion expressions and calculates, for each emotion expression, a mood index for each emotion expression by multiplying the proportion of each of the plurality of emotion expressions by a predetermined weight with respect to each of the plurality of emotion expressions, and
the mood level is based on a value obtained by dividing a maximum mood index being a largest mood index among mood indexes of the emotion expressions by a total value of the mood indexes of the emotion expressions.

Preferably, the plurality of emotion expressions are happy, surprise, neutral, fear, angry, disgust, and sad in Russell's circumplex model of affect.

Preferably, the data managing section acquires environmental data at least including air temperature and humidity in addition to the data related to the voice, the facial expression image, and the pulse wave of the subject, and
the emotion expression engine section adjusts each value of the brain fatigue level, the mood level, and the stress level by respectively multiplying the brain fatigue level, the mood level, and the stress level by a weight coefficient determined based on a discomfort index calculated from the air temperature and the humidity included in the environmental data.

Preferably, the data managing section acquires questionnaire data including a score of a stress check result of the subject in addition to the data related to the voice, the facial expression image, and the pulse wave of the subject, and
the emotion expression engine section adjusts each value of the brain fatigue level, the mood level, and the stress level by respectively multiplying the brain fatigue level, the mood level, and the stress level by a weight coefficient determined in accordance with the score included in the questionnaire data.

An information processing method is executed in a server connectable to a terminal device of a subject via a network, the information processing method including the steps of:
acquiring at least data related to voice, a facial expression image, and a pulse wave of the subject from the terminal device;
calculating a brain fatigue level based on a frequency of the voice, calculating a mood level by extracting an emotion of the subject from the facial expression image, and calculating a stress level by performing a frequency analysis of the pulse wave by fast Fourier transform and extracting a high-frequency section and a low-frequency section; and
displaying a graph of points plotted at coordinates corresponding to the brain fatigue level, the mood level, and the stress level in a three-dimensional space defined by an X-axis, a Y-axis, and a Z-axis, wherein
the data related to the voice is acquired by making an audio recording of at least a part of a video call with the subject via the terminal device,
the data related to the facial expression image is acquired by making a video recording of at least a part of a video call with the subject via the terminal device, and
the data related to the pulse wave is acquired via the terminal device from a pulse wave meter that measures a pulse wave of the subject.

An information processing system includes:
the information processing device; and
a terminal device capable of accessing the information processing device via a network, wherein
the terminal device transmits at least the data related to the voice, the data related to the facial expression image, and the data related to the pulse wave to the information processing device, and
the information processing device receives the data related to the voice, the data related to the facial expression image, and the data related to the pulse wave, transmits the brain fatigue level, the mood level, and the stress level calculated based on the respective pieces of received data to the terminal device, and displays, on the terminal device, a graph of points plotted at coordinates corresponding to the brain fatigue level, the mood level, and the stress level in a three-dimensional space defined by an X-axis, a Y-axis, and a Z-axis.

The program causes, by being executed by a computer, the computer to function as each section of the information processing device.

The program causes, by being executed by a computer, the computer to execute each step of the information processing method.

We thus provide an information processing system and the like capable of acquiring not only quantitative data such as a pulse wave/heart rate acquired from a pulse wave meter but also data of a stress check result and data such as voice, a facial expression image or the like of the subject during counseling using a video call, calculating values of a stress level, a brain fatigue level, and a mood level from the pieces of data and, by plotting the calculated values in a three-dimensional space defined by an X-axis, a Y-axis, and a Z-axis, visualizing the subject's emotion and the like and enabling diagnosis and treatment to be assisted, thereby realizing analysis of emotion of the subject with high accuracy, realizing early detection of mental illness, and contributing towards solving social problems.

### BRIEF DESCRIPTION OF THE DRAWINGS

FIG. 1 is a diagram showing an example of a configuration of an information processing system.

FIG. 2 is a block diagram showing an example of a hardware configuration of an information processing device.

FIG. 3 is a block diagram showing a configuration of the information processing device.

FIG. 4 is a diagram showing, in a table format, an example of data stored in a user information database of the information processing device shown in FIG. 3.

FIG. 5 is a flow chart showing a flow of processing for collecting various kinds of data from a terminal device of a subject.

FIG. 6 is a diagram showing an example of a user interface for performing a stress check using a questionnaire.

FIG. 7 is a diagram showing an example of a user interface for prompting a user to perform user registration after a stress check.

FIG. 8 is a diagram showing an example of a screen displaying a result of a stress check by a radar chart.

FIG. 9 is a diagram showing an example of a screen displaying a comment regarding a result of a stress check.

FIG. 10 is a diagram showing how a pulse wave/heart rate is measured from a fingertip of a subject using a pulse wave meter.

FIG. 11 is a diagram showing an example of a screen display on a terminal device of a subject when measuring a pulse wave/heart rate of the subject by a pulse wave meter.

FIG. 12 is a diagram showing an example of a screen display for acquiring an image of a facial expression of a subject from a video call between a counselor and the subject during counseling.

FIG. 13 is a diagram showing an example of a screen display for acquiring voice of a subject from a video call between a counselor and the subject during counseling.

FIG. 14 is a schematic diagram showing a configuration of an emotion expression engine section which calculates various indexes representing brain fatigue, mood, and stress from various kinds of collected data.

FIG. 15 is a diagram explaining weighting determined based on Russell's circumplex model of affect.

FIG. 16 is a diagram showing an example of calculating a mood level from various kinds of emotion expressions obtained based on Russell's circumplex model of affect.

FIG. 17 is a diagram showing an example of a numerical value conversion for plotting obtained values of various indexes representing brain fatigue, mood, and stress in a space defined by three axes.

FIG. 18 is a diagram showing an example of a graph display of a three-dimensional space defined by an X-axis, a Y-axis, and a Z-axis in which a change in emotion of a given subject obtained by an emotion expression engine is plotted in a time series.

FIG. 19 is a diagram showing an example of a graph display of a three-dimensional space defined by an X-axis, a Y-axis, and a Z-axis in which a change in emotion of another subject obtained by an emotion expression engine is plotted in a time series.

FIG. 20 is a diagram showing an example of per-type classification categories defined in a three-dimensional space defined by a tension axis (X-axis), a brain fatigue axis (Y-axis), and a mood axis (Z-axis).

### REFERENCE SIGNS LIST

10 information processing device
11, 21 CPU
11, 22 memory
13, 23 bus
14, 24 input/output interface
15, 25 input section
16, 26 output section
17, 27 storage section
18, 28 communicating section
20-1, 20-2, 20-n terminal device
30 pulse wave meter
32 body part
34 waveform display section
36 measuring section
111 emotion expression engine section
111A emotion expression core section
111B weight multiplication unit section
112 three-axes processing section
113 data managing section
171 user information database

### DETAILED DESCRIPTION

Hereinafter, examples will be described with reference to the accompanying drawings. The following examples describe our devices, systems and methods and are not intended to solely limit this disclosure to the examples. In addition, various modifications may be made without departing from the scope thereof. Furthermore, same constituent elements in the drawings will be denoted by same reference signs whenever possible and redundant descriptions will not be repeated.

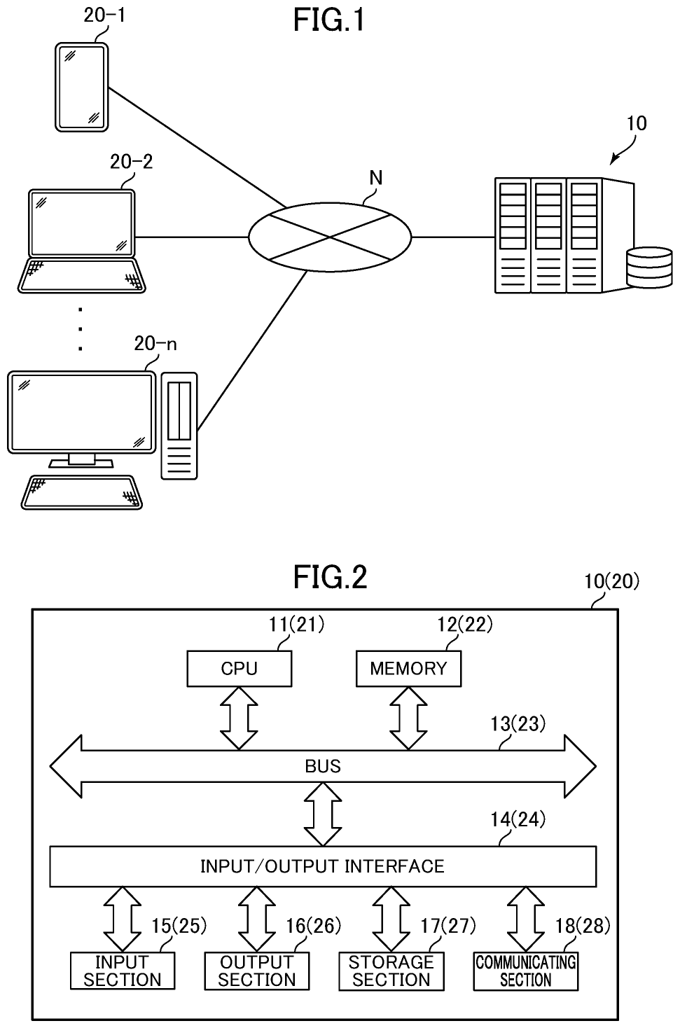

FIG. 1 shows an example of a configuration of an information processing system. Illustratively, the information processing system for visualizing emotion of a subject includes an information processing device 10 and n-number of (where n is any integer value equal to or larger than 1) terminal devices 20-n. In the drawing, terminal devices 20-1, 20-2 to a terminal device 20-n are illustrated as the n-number of terminal devices. However, in the following description, when the n-number of terminal devices are to be described without distinguishing the terminal devices from one another, reference signs will be partially omitted and the terminal devices will be simply referred to as a “terminal device 20.”

For example, the information processing device 10 is a computer such as a server that is connectable to a network N. In addition, for example, the terminal device 20 is a terminal connectable to the network N such as a personal computer, a notebook personal computer, a smartphone, or a mobile phone.

For example, the network N may be an open network such as the Internet or a closed network such as an intranet that is connected by a dedicated line. The network N is not limited thereto and, when appropriate, a closed network and an open network may be used in combination in accordance with a required security level or the like.

The information processing device 10 and the terminal device 20 are connected to the network N and are capable of communicating with each other. Using the terminal device 20, a subject (user) can access the information processing device 10 and transmit an answered questionnaire (medical questionnaire) of a stress check to the information processing device 10. The questionnaire of the stress check is, for example, a questionnaire of a stress check in the Stress Check Implementation Program issued by the Ministry of Health, Labour and Welfare (Stress Check System Introduction Manual—Ministry of Health, Labour and Welfare (URL https://www.mhlw.go.jp/bunya/roudokijun/anzeneisei12/pdf/150709-1.pdf) (Retrieved February 2021)).

In addition, to receive counseling from a counselor, the subject can perform a video call (video chat) with the counselor via the terminal device 20. Furthermore, the terminal device can transmit data related to a pulse wave of the subject having been measured using a pulse wave meter to the information processing device 10. As the pulse wave meter, for example, a device that measures a pulse wave from a fingertip of the subject can be used (Checking Corona-related Stress by a Fingertip, Jointly-developed by Yamagata University, Jul. 18, 2020, Asahi Shimbun Digital (URL https://www.asahi.com/articles/ASN7K6V99N78UZHB00M.html) (Retrieved Feb. 15, 2021)).

The information processing device 10 can acquire at least data related to voice, a facial expression image, and a pulse wave of the subject from the terminal device 20 and can calculate, based on the pieces of data, indexes representing an emotion (mental status) of the subject such as a brain fatigue level, a mood level, and a stress level to be described later.

FIG. 2 is a block diagram showing an example of a hardware configuration of an information processing device. In the drawing, reference signs corresponding to hardware of the information processing device 10 are described without parentheses. In addition, since a hardware configuration of the terminal device 20 is similar to that of the information processing device 10, reference signs corresponding to hardware of the terminal device 20 are described with parentheses.

For example, the information processing device 10 is a server (computer) and, illustratively, the information processing device 10 includes a CPU (Central Processing Unit) 11, a memory 12 constituted of a ROM (Read Only Memory), a RAM (Random Access Memory) and the like, a bus 13, an input/output interface 14, an input section 15, an output section 16, a storage section 17, and a communicating section 18.

The CPU 11 executes various kinds of processing in accordance with a program recorded in the memory 12 or a program loaded to the memory 12 from the storage section 17. For example, the CPU 11 can execute a program that causes the server (computer) to function as an information processing device capable of visualizing emotion of the subject and assisting diagnosis and treatment. In addition, at least a part of the functions of the information processing device can be implemented by hardware with an application specific integrated circuit (ASIC) or the like.

The memory 12 also stores, when appropriate, data and the like necessary for the CPU 11 to execute the various kinds of processing. The CPU 11 and the memory 12 are connected to each other via the bus 13. The input/output interface 14 is also connected to the bus 13. The input section 15, the output section 16, the storage section 17, and the communicating section 18 are connected to the input/output interface 14.

The input section 15 can be realized by an input device such as a keyboard or a mouse independent of a main body that houses other sections of the information processing device 10 and various kinds of information can be input in accordance with an instruction operation by a user (manager) or the like of the information processing device 10. The input section 15 may be constituted of various buttons, a touch panel, a microphone or the like.

The output section 16 is constituted of a display, a speaker or the like and outputs data related to a text, a still image, a moving image, voice or the like. The text data, still image data, moving image data, voice data or the like outputted by the output section 16 is outputted from the display, the speaker or the like to be recognizable by the user as characters, an image, video, or voice.

The storage section 17 is constituted of a storage device such as a DRAM (Dynamic Random Access Memory) or another semiconductor memory, a solid state drive (SSD), or a hard disk and is capable of storing various kinds of data.

The communicating section 18 realizes communication to be performed with other devices. For example, the communicating section 18 is capable of communicating with other devices (for example, the terminal devices 20-1, 20-2 to 20-n) and with one another via the network N.

Although not illustrated, the information processing device 10 is appropriately provided with a drive when necessary. For example, a removable medium constituted of a magnetic disk, an optical disk, a magneto optical disk, a semiconductor memory or the like is appropriately mounted to the drive. The removable medium stores a program for realizing a function of visualizing emotion or the like of the subject by calculating values of a stress level, a brain fatigue level, and a mood level of the subject and plotting the values in a three-dimensional space defined by an X-axis, a Y-axis, and a Z-axis and various kinds of data such as text data and image data. A program read from the removable medium by the drive and the various kinds of data are installed in the storage section 17 when necessary.

Next, a configuration of hardware of the terminal device 20 will be described. As shown in FIG. 2, illustratively, the terminal device 20 includes a CPU 21, a memory 22, a bus 23, an input/output interface 24, an input section 25, an output section 26, a storage section 27, and a communicating section 28. Each of these sections has a similar function to each section which is included in the information processing device 10 and which has the same name but which solely differs in its reference sign. Therefore, overlapping descriptions will be omitted. When the terminal device 20 is configured as a mobile device, each piece of hardware included in the terminal device 20 and a display or a speaker may be realized as an integrated device.

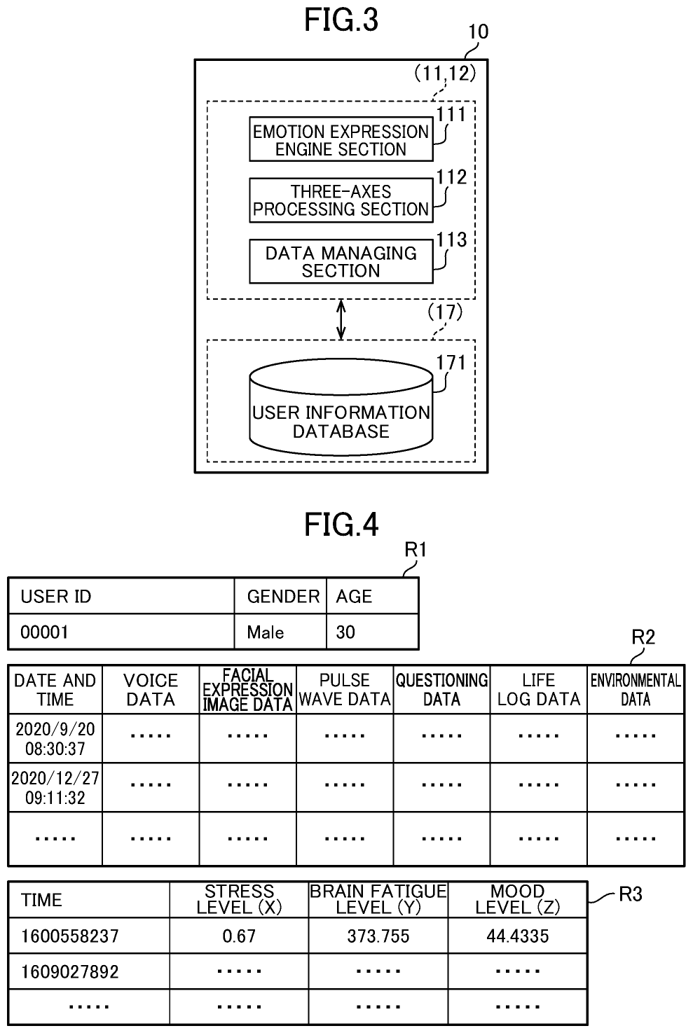

A functional configuration of the information processing device 10 included in the information processing system to visualize emotion of the subject will be described with reference to FIGS. 2 and 3. FIG. 3 is a block diagram showing a configuration of the information processing device according to the example. For example, when a server (computer) executes a program for performing processing such as: acquiring at least data related to voice, a facial expression image, and a pulse wave of the subject from the terminal device 20; calculating a brain fatigue level based on a frequency of the voice; calculating a mood level by extracting an emotion of the subject from the facial expression image; calculating a stress level by performing a frequency analysis of the pulse wave by fast Fourier transform and extracting a high-frequency section and a low-frequency section; and displaying a graph of points plotted at coordinates corresponding to the brain fatigue level, the mood level, and the stress level in a three-dimensional space defined by an X-axis, a Y-axis, and a Z-axis, illustratively, the server functions as the information processing device 10 and at least an emotion expression engine section 111, a three-axes processing section 112, and a data managing section 113 function in hardware resources including the CPU 11, the memory 12 and the like.

In addition, using a partial storage area of the storage section 17, the storage section 17 can be caused to function as a user information database 171. As another example, the user information database 171 can be constituted of an external storage device separate from the information processing device 10 and, for example, a cloud storage can be used as the external storage device. While the user information database 171 is configured as a single storage device in these examples, the user information database 171 may be stored divided into two or more storage devices.

The emotion expression engine section 111 can calculate a brain fatigue level, a mood level, and a stress level as indexes that represent emotion of the subject based on data related to voice, a facial expression image, and a pulse wave of the subject acquired from the terminal device 20. For example, the emotion expression engine section 111 can calculate a brain fatigue level based on a frequency of the voice, calculate a mood level by extracting an emotion of the subject from the facial expression image, and calculate a stress level by performing a frequency analysis of the pulse wave by fast Fourier transform and extracting a high-frequency section and a low-frequency section.

The three-axes processing section 112 can generate a graph of points plotted at coordinates corresponding to the brain fatigue level, the mood level, and the stress level calculated by the emotion expression engine section 111 and display the graph on the information processing device 10 or the terminal device 20. In addition, the three-axes processing section 112 can generate a graph of points plotted according to a time series at coordinates corresponding to the brain fatigue level, the mood level, and the stress level of the subject for each of the dates and times at which data related to voice, a facial expression image, and a pulse wave of the subject has been acquired in a three-dimensional space defined by an X-axis, a Y-axis, and a Z-axis and display the graph on the information processing device 10 or the terminal device 20.

The data managing section 113 can acquire at least data related to voice, a facial expression image, and a pulse wave of the subject from the terminal device 20 and store the data in storage means (for example, the user information database 171) of the information processing device 10. In addition, the data managing section 113 can associate the data related to the voice, the facial expression image, and the pulse wave of the subject with a date and time at which the data has been acquired and store the associated data in the storage means (the user information database 171) of the information processing device.

FIG. 4 is a diagram showing, in a table format, an example of data stored in a user information database of the information processing device shown in FIG. 3. A table R1 stores a user ID which is information identifying a subject (user), a gender such as male, female, or other gender identity, and an age in association with one another. For example, the user information database 171 can store tables R2 and R3 in association with the user information in the table R1.

In addition, the table R2 stores, in association with a date and time, voice data, facial expression image data, pulse wave data, questionnaire data including answers to a stress check by the subject, life log data that records behavior and the like of the subject, and environmental data including air temperature and humidity received from the terminal device 20. The date and time included in the table R2 is the date and time at which data related to voice, a facial expression image, and a pulse wave of the subject had been received from the terminal device a date and time at which the subject had accessed the information processing device 10 using the terminal device 20 and the like.

Furthermore, in association with time obtained by normalizing a date and time stored in the table R2, the stress level, the brain fatigue level, and the mood level of the subject calculated by the emotion expression engine section 111 are stored as values of an X-axis, a Y-axis, and a Z-axis in a three-dimensional space. Normalization of a date and time can be realized by, for example, converting the date and time into a UNIX (registered trademark) time.

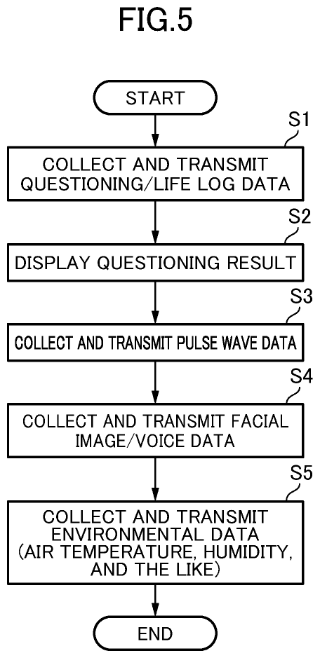

FIG. 5 is a flow chart showing a flow of processing for collecting various kinds of data from a terminal device of a subject. For example, the processing is executed by the terminal device 20. The terminal device 20 collects questionnaire data including the subject's answers to a stress check questionnaire and transmits the questionnaire data to the information processing device 10 (step S1). In addition, in step S1, life log data which records daily life, activities, behavior and the like of the subject can be collected together with the questionnaire data and transmitted to the information processing device 10.

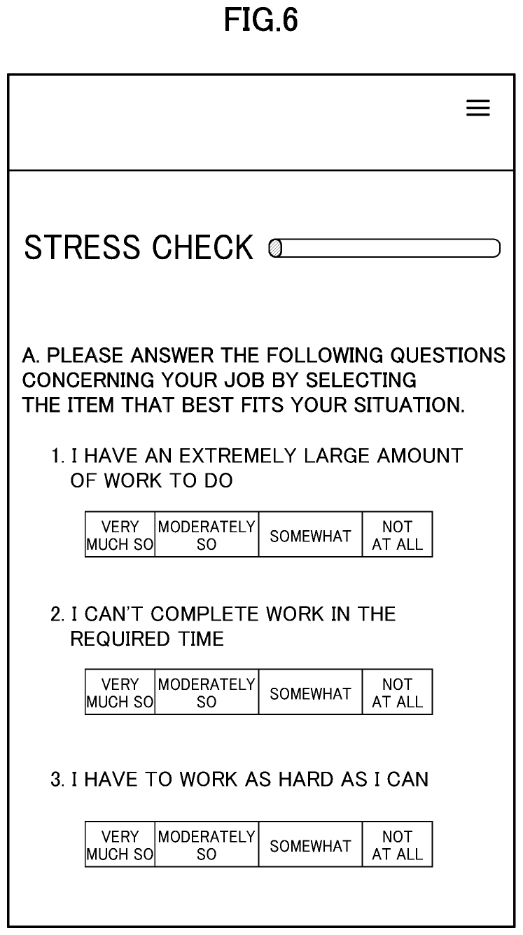

Subsequently, the terminal device 20 displays a questioning result on a screen (step S2). Screen displays of the terminal device 20 when executing processing from step S1 to step S2 are shown in FIGS. 6 to 9.

FIG. 6 shows an example of a user interface for performing a stress check using a questionnaire. This is an example of a questionnaire of a stress check that is displayed on the screen of the terminal device 20. Under a message that reads “A. Please answer the following questions concerning your job by selecting the item that best fits your situation,” a question that reads “1. I have an extremely large amount of work to do” is displayed. By performing a selection operation such as clicking or tapping of any option among “Very much so,” “Moderately so,” “Somewhat” and “Not at all,” the subject can select the option. A similar description applies to the other questions. The number of questions can be set to, for example, 57 items of the stress check questionnaire issued by the Ministry of Health, Labour and Welfare (Stress Check System Introduction Manual—Ministry of Health, Labour and Welfare (URL https://www.mhlw.go.jp/bunya/roudokijun/anzeneisei12/pdf/150709-1.pdf) (Retrieved Feb. 15, 2021)).

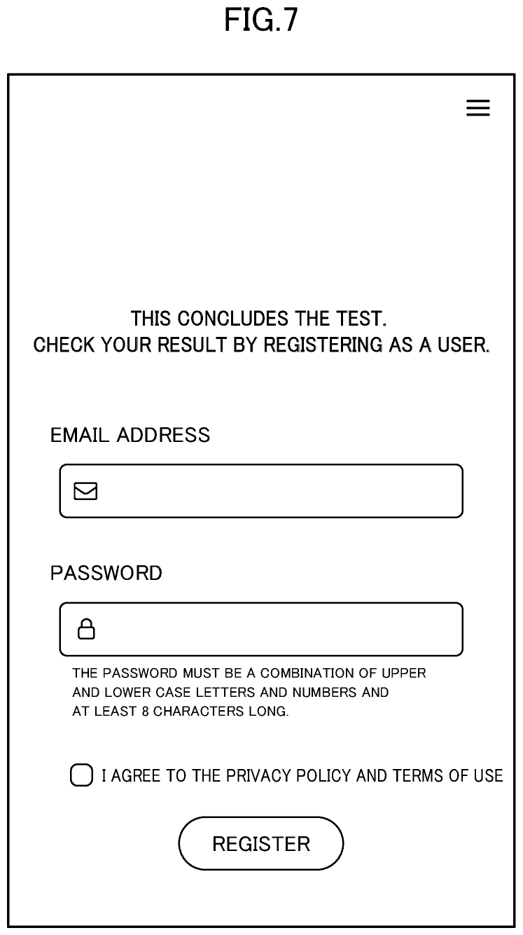

After the subject answers all of the items of the stress check questionnaire, contents such as those shown in FIG. 7 are displayed on the screen of the terminal device 20. FIG. 7 shows an example of a user interface for prompting a user to perform user registration after the stress check. According to a message that reads “This concludes the test. Check your result by registering as a user,” for example, the subject registers information on the subject by inputting an email address, a password, and other necessary items and pressing a register button in a bottom part of the screen.

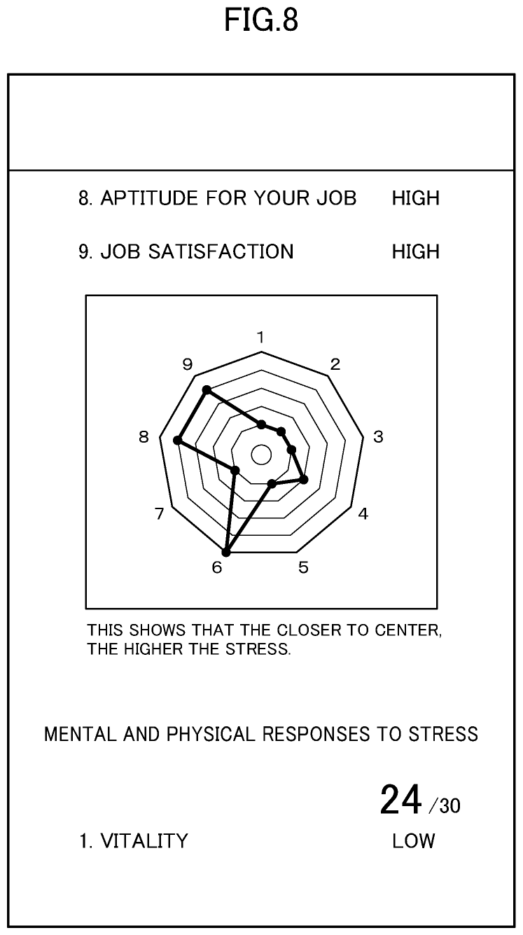

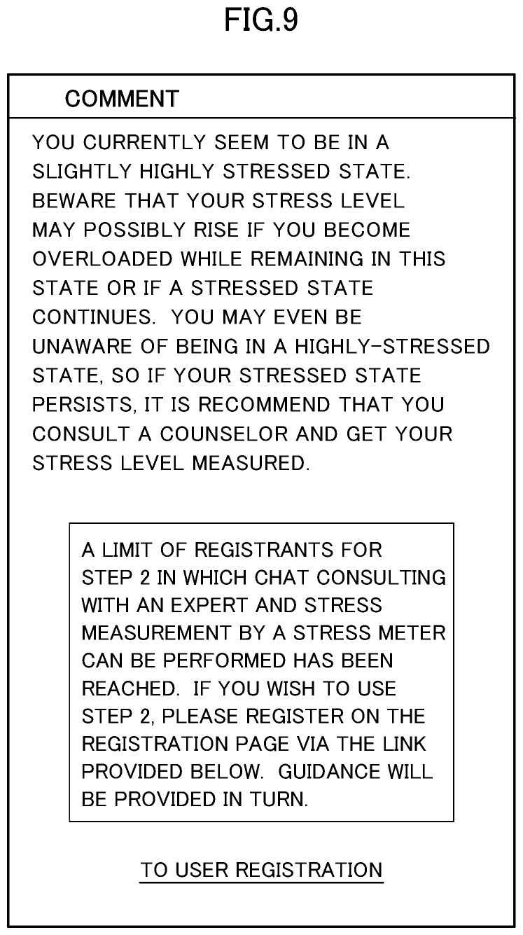

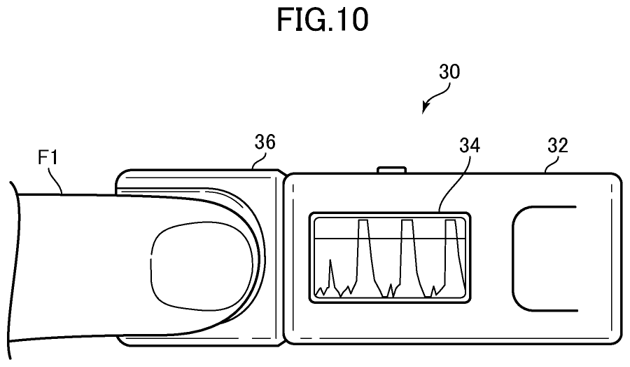

After registration, for example, contents such as those shown in FIG. 8 are displayed on the screen of the terminal device 20. FIG. 8 shows an example of a screen displaying a result of a stress check by a radar chart. The radar chart indicates that, the closer to a center of the radar chart, the higher the stress of the subject. FIG. 9 shows an example of a screen displaying a comment regarding a result of a stress check. The terminal device 20 can display a comment by a counselor (expert) in accordance with a questioning result of the subject such as “You currently seem to be in a slightly highly stressed state . . . ” in an upper half of the screen.

In addition, the terminal device 20 can display a message such as “A limit of registrants for step S2 in which chat consulting with an expert and stress measurement by a stress meter can be performed has been reached. If you wish to use step S2, please register on the registration page via the link provided below” in a lower half of the screen and can prompt the subject to register more detailed personal information by performing a selection operation such as a click or a tap of “To user registration” at the bottom of the screen. The personal information of the subject having been transmitted from the terminal device 20 to the information processing device 10 is stored by the data managing section 113 in, for example, the user information database.

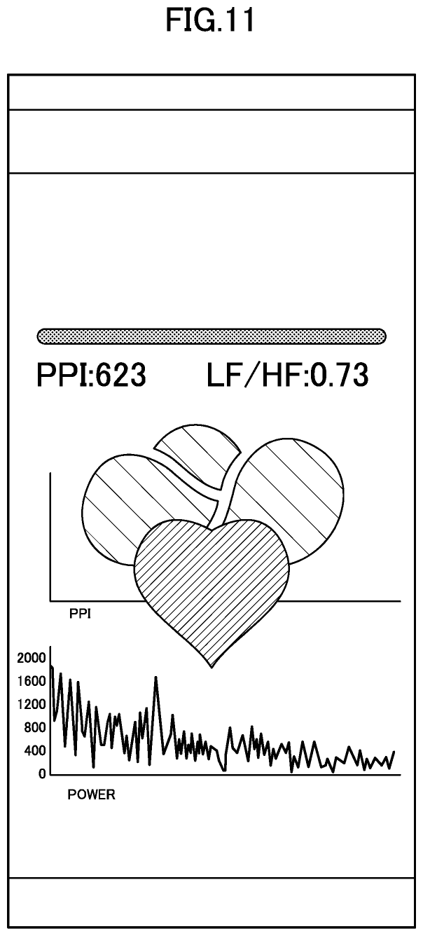

Once again referring to the flow chart shown in FIG. 5, after step S2, the terminal device 20 collects pulse wave data from the pulse wave meter that measures a pulse wave (heart rate) of the subject and transmits the pulse wave data to the information processing device 10 (step S3). FIG. 10 shows how a pulse wave/heart rate is measured from a fingertip of a subject using the pulse wave meter. A pulse wave meter 30 includes a body part 32, a waveform display section 34 provided in the body part 32, and a measuring section 36. By the subject pressing a fingertip against the measuring section 36 of the pulse wave meter 30, the pulse wave meter 30 can measure a pulse wave/heart rate of the subject and a waveform based on the pulse wave/heart rate is displayed on the waveform display section 34 provided in the body part 32. A measurement time by the pulse wave meter 30 can be set to, for example, 180 seconds (3 minutes) per one measurement.

The terminal device 20 can communicably connect to the pulse wave meter 30 and receive pulse wave data of the subject from the pulse wave meter 30. FIG. 11 shows an example of a screen display on a terminal device of a subject when measuring a pulse wave/heart rate of the subject by a pulse wave meter. For example, the terminal device 20 can display a waveform based on pulse wave data of the subject on the screen and can also display a pulse interval (Peak-to-Peak Interval: PPI) that is an interval from a peak to a next peak of the pulse wave of one heartbeat, a value of low-frequency section (Low Frequency: LF)/high-frequency section (High Frequency: HF) of the pulse wave data and the like on the screen.

Referring to the flow chart shown in FIG. 5, after step S3, by making a video recording and an audio recording of at least a part of a video call (video chat) with a counselor or an expert, the terminal device 20 acquires data related to a facial expression image and data related to voice of the subject (step S4). The measurement of the pulse wave of the subject by the pulse wave meter in step S3 can be continuously performed and the terminal device 20 can collect pulse wave data even when a video call is in progress. After step S4, the terminal device 20 can collect environmental data including air temperature, humidity and the like and transmit the environmental data to the information processing device 10 (step S5). Processing of steps S4 and S5 can be performed in the information processing device 10 instead of the terminal device 20.

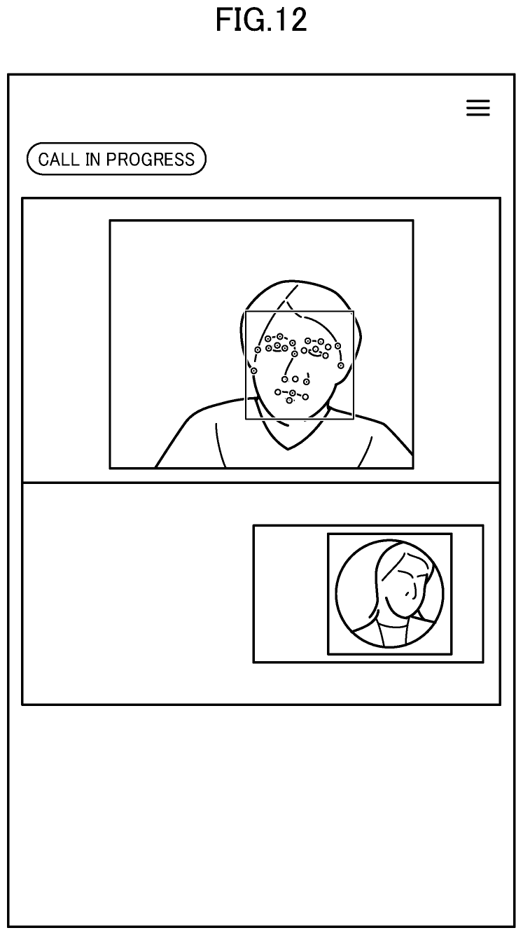

FIG. 12 shows an example of a screen display that acquires an image of a facial expression of a subject from a video call between a counselor and the subject during counseling. A facial expression of the subject is projected on an upper side of the screen shown in FIG. 12 and a counselor is projected on a lower side of the screen. To recognize the facial expression of the subject, a video recording of the video call during counseling is made in the terminal device 20 or the information processing device 10 until at least a predetermined video recording time (for example, 15 minutes) is reached. In other words, data related to the facial expression image of the subject is data acquired by making a continuous video recording of a moving image of a facial expression of the subject until at least a predetermined video recording time is reached during a video call with the subject via the terminal device 20.

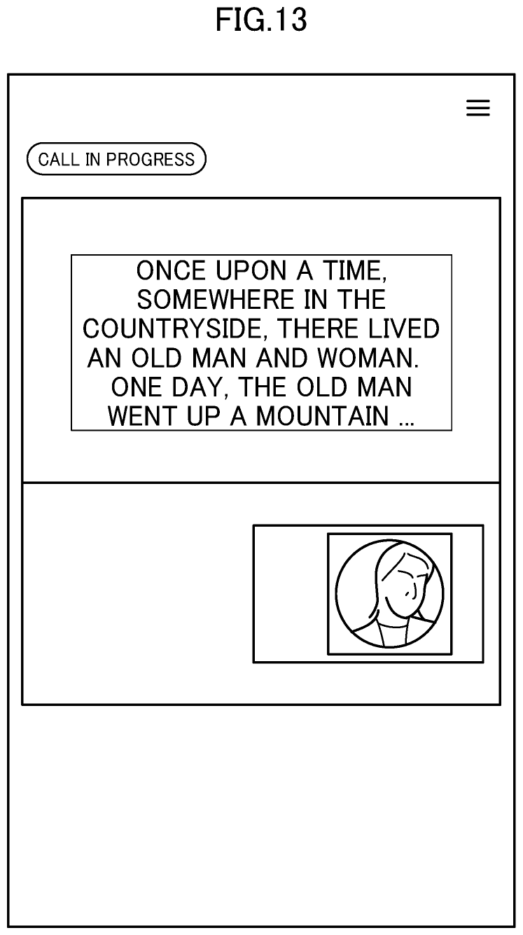

FIG. 13 shows an example of a screen display for acquiring voice of a subject from a video call between a counselor and the subject during counseling. Fixed phrases (for example, “Once upon a time, somewhere in the countryside, there lived an old man and woman. One day, the old man went up a mountain . . . ”) to be read out loud by the subject is displayed on an upper side of the screen shown in FIG. 13 and a counselor is projected on a lower side of the screen. Due to the subject reading the fixed phrases out loud, an audio recording of the voice of the oral reading of the fixed phrases is made until at least a predetermined audio recording time (for example, around 40 seconds) is reached in the terminal device 20 or the information processing device 10. In other words, the data related to the voice is data acquired by making a continuous audio recording of the voice of the subject reading out loud predetermined fixed phrases displayed on the terminal device 20 at least until a predetermined audio recording time is reached during a video call with the subject via the terminal device 20.

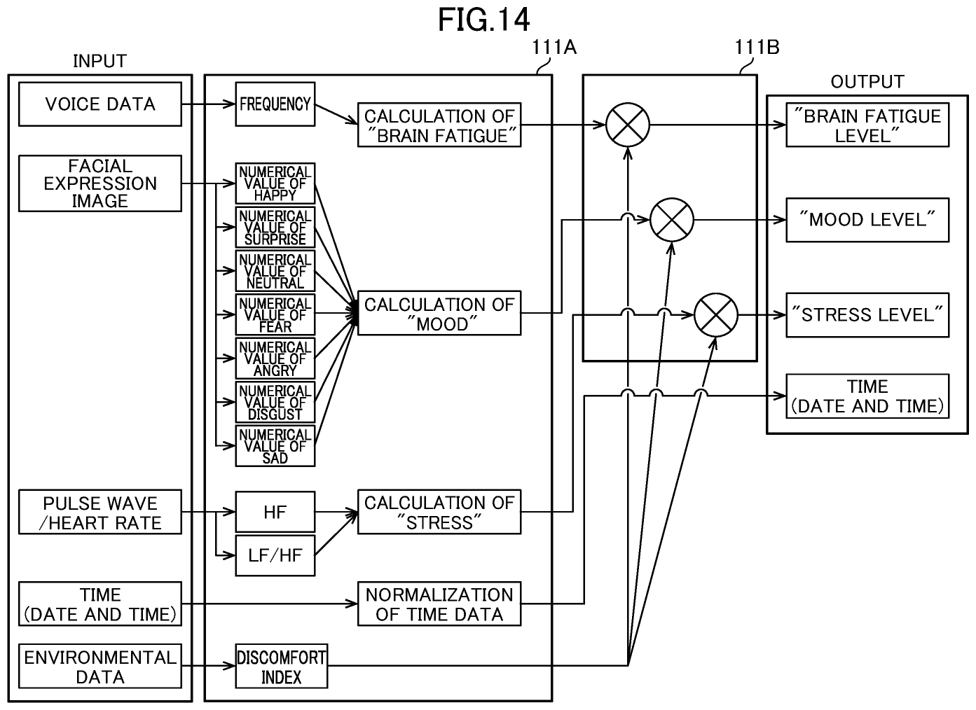

FIG. 14 is a schematic diagram showing a configuration of an emotion expression engine section which calculates various indexes representing brain fatigue, mood, and stress from various kinds of collected data. The emotion expression engine section 111 can be functionally divided into an emotion expression core section 111A and a weight multiplication unit section 111B. The emotion expression core section 111A can calculate a brain fatigue level, a mood level, and a stress level that are quantitative indexes related to brain fatigue, mood, and stress. In addition, the emotion expression core section 111A can normalize a date and time, calculate a discomfort index and the like.

The weight multiplication unit section 111B can adjust each value of the brain fatigue level, the mood level, and the stress level calculated by the emotion expression core section 111A by respectively multiplying the brain fatigue level, the mood level, and the stress level by a weight coefficient determined based on a discomfort index calculated from the air temperature and the humidity included in the environmental data. When the discomfort index is denoted by DI, the air temperature by T, and the humidity by H, for example, the discomfort index can be obtained by a formula expressed as DI=0.81T+0.01H×(0.99T−14.3)+46.3. In addition, although not illustrated in FIG. 14, the weight multiplication unit section 111B can acquire questionnaire data including a score of a stress check result of the subject and adjust each value of the brain fatigue level, the mood level, and the stress level calculated by the emotion expression core section 111A by respectively multiplying the brain fatigue level, the mood level, and the stress level by a weight coefficient determined in accordance with the score included in the questionnaire data.

The emotion expression engine section 111 calculates a brain fatigue level in the emotion expression core section 111A from voice data of the subject acquired as input data. For example, the brain fatigue level can be obtained by calculating a CEM (Cerebral Exponent Macro) value that represents a cerebral activity index. A cerebral activity index measurement algorithm (SiCECA algorithm, Yuki Aoki et al., Development of Fatigue Degree Estimation System for Smartphone, E-037 FIT2013) developed by the Electronic Navigation Research Institute enables a cerebral activity index (CEM value) to be calculated from voice. By executing the cerebral activity index measurement algorithm, the emotion expression engine section 111 can acquire one or more (for example, around two to five) CEM values for each subject from data related to the voice of the subject. For example, the brain fatigue level corresponds to a value obtained by calculating an average value of the one or more CEM values.

FIG. 17 shows an example of a numerical value conversion for plotting obtained values of various indexes representing brain fatigue, mood, and stress in a space defined by three axes. In the example shown in FIG. 17, four CEM values 431.08, 360.73, 342.76, and 360.45 are acquired by the cerebral activity index measurement algorithm and the brain fatigue level is 373.755 being an average value of the CEM values.

Once again referring to FIG. 14, the emotion expression engine section 111 calculates a mood level in the emotion expression core section 111A from facial expression image data of the subject acquired as input data. For example, the mood level is determined based on a count of a plurality of emotion expressions recognized from a moving image of a facial expression of the subject based on a facial expression recognition algorithm. As the facial expression recognition algorithm, an algorithm according to “Face classification and detection” (Face classification and detection (URL https://github.com/oarriaga/face_classification) (Retrieved Feb. 15, 2021)) that is open source software can be used.

By executing the facial expression recognition algorithm, the emotion expression engine section 111 can recognize a plurality of emotion expressions from a moving image of a facial expression of the subject included in the data related to the facial expression image. For example, the plurality of emotion expressions may be the seven kinds, namely, happy, surprise, neutral, fear, angry, disgust, and sad in Russell's circumplex model of affect (J. A. Russell et al., Core affect, prototypical emotional episodes, and other things called emotion: Dissecting the elephant, Journal of Personality and Social Psychology, 76(5), 805-819)).

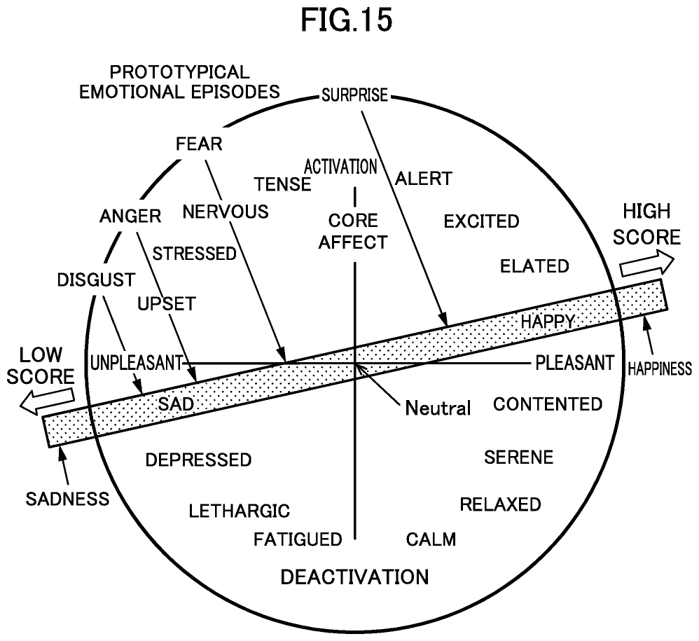

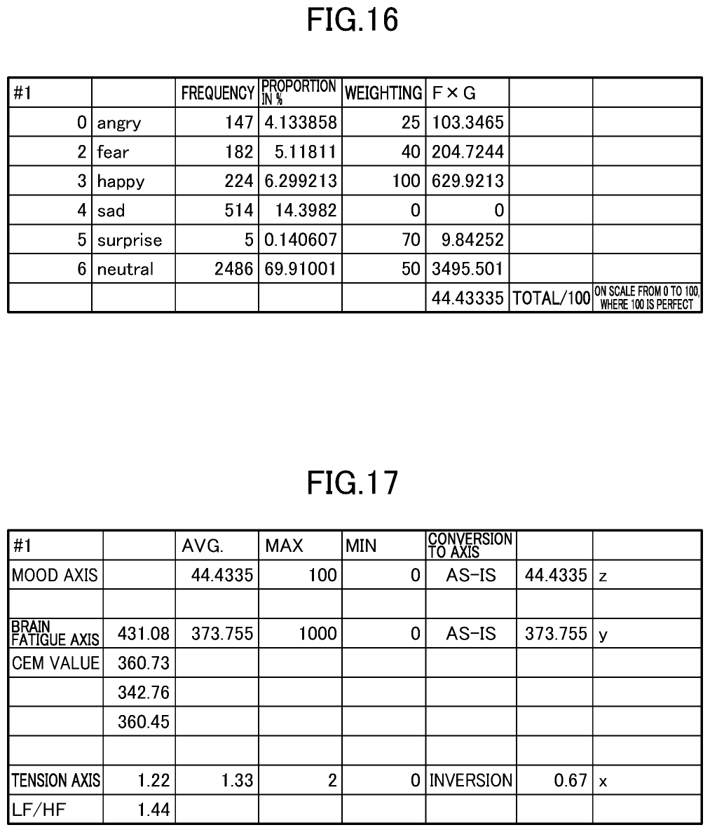

By executing the facial expression recognition algorithm (for example, the open source software “Face classification and detection”), the emotion expression engine section 111 counts each of the plurality of emotion expressions recognized from a moving image of a facial expression of the subject included in the data related to the facial expression image. The emotion expression engine section 111 calculates a proportion for each of the plurality of emotion expressions and calculates, for each emotion expression, a mood index for each emotion expression by multiplying the proportion of each of the plurality of emotion expressions by a predetermined weight with respect to each of the plurality of emotion expressions. The predetermined weight with respect to each of the plurality of emotion expressions can be determined based on Russell's circumplex model of affect as shown in FIG. 15 and, for example, the weighting shown in FIG. 16 may be determined.

FIG. 15 is a diagram for explaining weighting that is determined based on Russell's circumplex model of affect. FIG. 16 shows an example of calculating a mood level from various kinds of emotion expressions obtained based on Russell's circumplex model of affect. In the predetermined weight with respect to each of the plurality of emotion expressions, a magnitude of a weight coefficient can be adjusted in an order of happy, surprise, neutral, fear, angry, disgust, and sad. Referring to FIG. 16, for example, a weighting coefficient (weight coefficient) of happy can be set to 100, a weighting coefficient of surprise to 70, a weighting coefficient of neutral to 50, and so on.

In the example shown in FIG. 16, among proportions of the plurality of emotion expressions, neutral indicates a highest proportion at 69.91001, followed by happy with a proportion of 6.299213. By multiplying the proportion (F) 69.91001 of neutral by a predetermined weight coefficient (G) of 50, a value of a mood index F×G is calculated as 3495.501. In a similar manner, by multiplying the proportion (F) 6.299213 of happy by a predetermined weight coefficient (G) of 100, a value of a mood index F×G is calculated as 629.9213. In this manner, the emotion expression engine section 111 calculates, for each emotion expression, a mood index for each emotion expression by multiplying the proportion of each of the plurality of emotion expressions by a predetermined weight with respect to each of the plurality of emotion expressions.

The emotion expression engine section 111 can adopt a value obtained by dividing a maximum mood index being a largest mood index among mood indexes of the emotion expressions by a total value of the mood indexes of the emotion expressions as the mood level. In the example shown in FIG. 16, the mood index 3495.501 of neutral is the maximum mood index. The mood level is a value calculated by dividing the maximum mood index (3495.501) by a total value (4443.33572) of the mood indexes of the emotion expressions: 3495.501/4443.33572=44.43335. In the example shown in FIG. 16, display of disgust is omitted since there is no value related to disgust.

Once again referring to FIG. 14, the emotion expression engine section 111 calculates a stress level in the emotion expression core section 111A from data related to a pulse wave (heart rate) of the subject acquired as input data. The data related to the pulse wave is data acquired by dividing a pulse wave measured by the pulse wave meter 30 into sections, each section being a predetermined time interval (for example, 180 seconds).

The emotion expression engine section 111 divides, for each section of the pulse wave, the pulse wave in the section into Hamming windows and calculates, with respect to the pulse wave in each of the Hamming windows, a pulse interval PPI being an interval from a peak to a next peak of the pulse wave of one heartbeat and a time of day. The emotion expression engine section 111 generates, for each section of the pulse wave, a time-PPI graph which plots a point at coordinates corresponding to the pulse interval PPI and the time of day in a two-dimensional space defined by time of day as an axis of abscissa and PPI as an axis of ordinate. By performing interpolation such as linear interpolation or cubic spline interpolation between discrete values in the time domain-PPI graph, subsequently applying a fast Fourier transform FFT, and respectively integrating a power spectral density PSD of a result of the FFT in a low-frequency section and in a high-frequency section, the emotion expression engine section 111 can calculate an LF value corresponding to a low-frequency component, an HF value corresponding to a high-frequency component, and an LF/HF value. Known methods of calculating the LF value, the HF value, and the LF/HF value from the pulse interval PPI include a stressed state estimation method described in J. A. Russell et al., Core affect, prototypical emotional episodes, and other things called emotion: Dissecting the elephant, Journal of Personality and Social Psychology, 76(5), 805-819).

The emotion expression engine section 111 can adopt the LF/HF value (sympathetic nervous system index) as the stress level. Alternatively, the stress level can be a level based on at least one value among the LF value, the HF value, and the LF/HF value. For example, referring to previous data values, normalization can be performed by setting a maximum value of LF/HF to 2 and, when also using HF (parasympathetic nervous system index), setting a maximum value of HF to 900. When there are a plurality of pieces of normalized data in each window section, an average value is calculated. In the example shown in FIG. 17, 1.22 and 1.44 are calculated as LF/HF values and an average of the LF/HF values is calculated as 1.33.

When only an LF/HF value is used as a stress level, inversion is performed so that a maximum value (MAX) of stress becomes minimum (MIN) and (MAX2—standard value) is converted into one axis. For example, in the example shown in FIG. 17, an axis of the stress level (tension axis) has a MAX value of 2 and a MIN value of 0, and inversion is performed when converting into an axis. Note that an axis of the mood level (mood axis) and an axis of the brain fatigue level (brain fatigue axis) is used as-is without inverting the axes. When an HF value is also used as a stress level, conversion into one axis is performed so that maximum stress becomes minimum and maximum relaxation becomes maximum via neutral.

The low-frequency section can be set from 0.04 Hz or higher to lower than 0.15 Hz and the high-frequency section can be set from 0.15 Hz or higher to lower than 0.4 Hz.

As shown in FIG. 14, the emotion expression engine section 111 can acquire time (date and time), environmental data and the like as input data in addition to data related to voice, a facial expression image, and a pulse (heart rate) of the subject. As described above, the time (date and time) is converted into time (for example, UNIX time) normalized by the emotion expression core section 111A, a discomfort index can be obtained from temperature and humidity included in the environmental data, and the brain fatigue level, the mood level, and the stress level can be respectively multiplied in the weight multiplication unit section 111B by a weight coefficient determined in advance in accordance with the discomfort index.

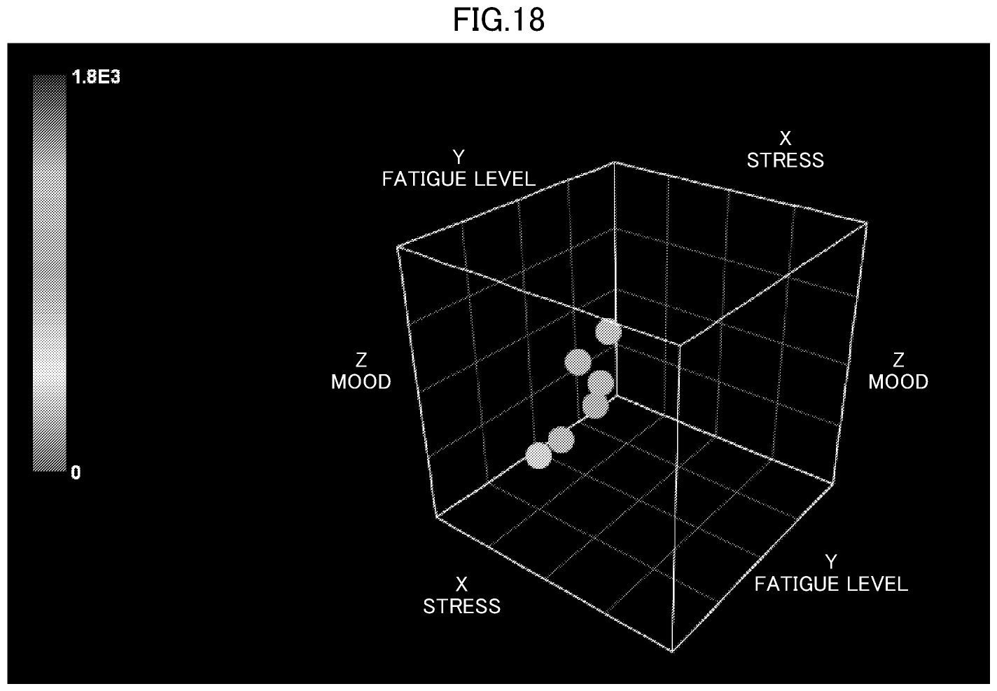

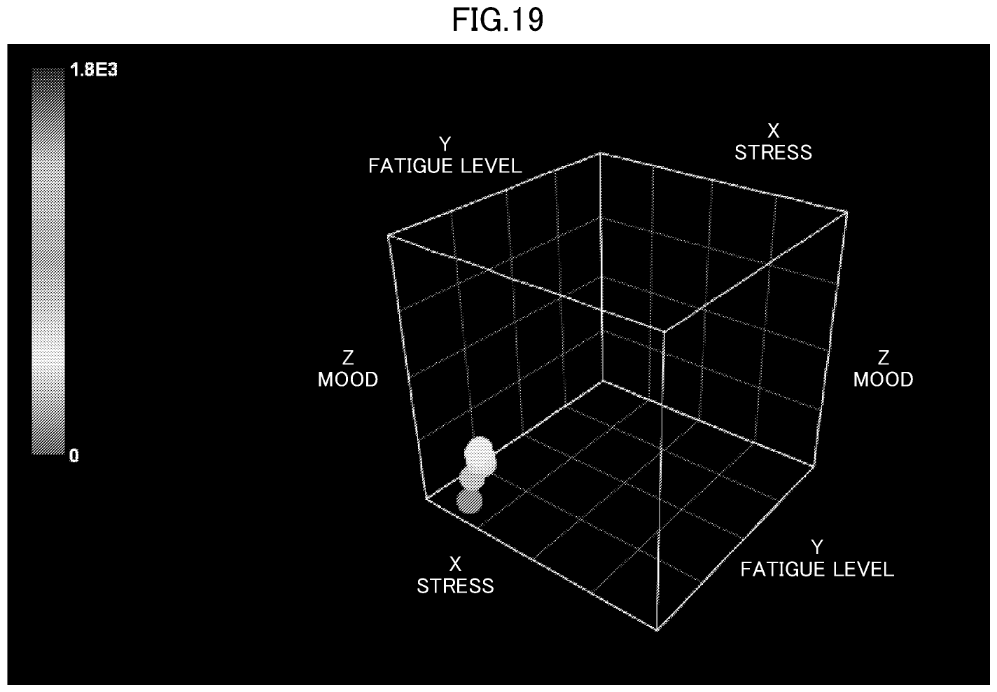

FIG. 18 shows an example of a graph display of a three-dimensional space defined by an X-axis, a Y-axis, and a Z-axis in which a change in emotion of a given subject obtained by an emotion expression engine is plotted in a time series. In addition, FIG. 19 shows an example of a graph display of a three-dimensional space defined by an X-axis, a Y-axis, and a Z-axis in which a change in emotion of another subject obtained by an emotion expression engine is plotted in a time series. The three-axes processing section 112 can generate and display a graph of points plotted at coordinates corresponding to the brain fatigue level, the mood level, and the stress level in a three-dimensional space defined by an X-axis, a Y-axis, and a Z-axis. In addition, the three-axes processing section 112 can display a graph of points plotted according to a time series at coordinates corresponding to the brain fatigue level, the mood level, and the stress level of the subject for each of the dates and times in the three-dimensional space. Accordingly, a change in emotion of the subject can be visualized and, therefore, an analysis of the emotion of the subject can be realized with high accuracy.

Such a graph display in a three-dimensional space can be analyzed in multiple dimensions by integrating the three axes of the brain fatigue level, the mood level, and the stress level with a time axis and is displayed to be readily interpretable for the subject, experts, and other users. While a diagnosis result is depicted by a radar chart or the like in conventional stress checks, a correspondence between factors is hardly represented. As shown in FIGS. 18 and 19, a correspondence with respect to a result can be displayed in a readily interpretable manner. Axes of the graph display in a three-dimensional space are appropriately interchangeable in accordance with information desired by a user. Measuring stress in a time series and classifying the stress into per-type categories (clusters) according to patterns (trends) enables future states to be predicted.

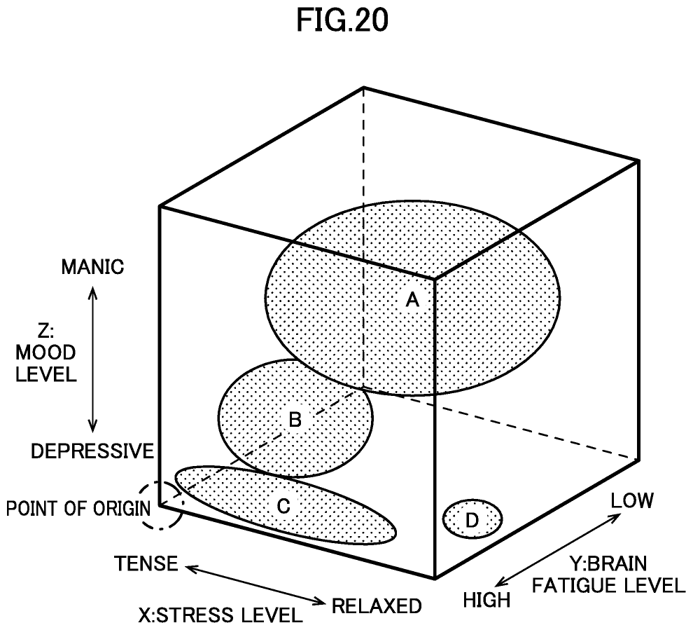

FIG. 20 shows an example of per-type classification categories set in a three-dimensional space defined by a tension axis (X-axis), a brain fatigue axis (Y-axis), and a mood axis (Z-axis). A bottom left corner represents a point of origin and the closer to the point of origin, stress (X-axis) is high, the brain fatigue level (Y-axis) is high, and the mood level (Z-axis) indicates a depressing tendency. A similar description applies to FIGS. 18 and 19. While a state where moods move from the point of origin can be confirmed in the example of a subject shown in FIG. 18, a continuation of a state where moods concentrate near the point of origin, stress (X-axis) is high, the brain fatigue level (Y-axis) is high, and the mood level (Z-axis) is depressed can be confirmed in the example of another subject shown in FIG. 19 and a response can be considered in advance when there is a risk of mental illness.

For example, the per-type classification categories defined in the three-dimensional space shown in FIG. 20 can be defined as shown in Table 1.

TABLE 1

| | |
|---|---|
| Type A: A healthy person | Emotions are neither in an extremely manic state nor an |
| | extremely depressive state and fluctuate in an intermediate range within |
| | a time period. The brain fatigue level repeats a pattern of becoming |
| | elevated when concentrating on work or study but recovering after rest. |
| | Autonomic nerves also repeat tension and relaxation. |
| Type B: A person with a mild | Somewhat close to the point of origin and fluctuation widths of mood, |
| risk of a mental disorder | brain fatigue level, and autonomic nerves are all limited. |
| Type C: A person with a | Close to the point of origin and fluctuation widths of mood, brain |
| risk of a mental disorder | fatigue level, and autonomic nerves are all extremely limited. |
| | Mood is always on the depressed side, and autonomic nerves |
| | are in a stressed state or there is a shortage of emotion such as being |
| | moved. |
| | Although the brain fatigue level is not high due to lack of |
| | motivation, this is a state where the brain is not awakened in a healthy |
| | manner. |
| Type D: (Omitted) | (Omitted) |

As shown in FIG. 20, the three-dimensional space is divided into a plurality of per-type classification categories, and the three-axes processing section 112 can notify the terminal device 20 of the subject, the information processing device 10 or the like of a category to which a point of coordinates corresponding to the brain fatigue level, the mood level, and the stress level of the subject belongs among the plurality of per-type classification categories in the three-dimensional space.

In addition, an improvement plan to be proposed to the subject is determined for each of the plurality of per-type categories, and the emotion expression engine section 111 can notify the terminal device 20 of the subject, the information processing device 10 or the like of the improvement plan with respect to the category to which a point of coordinates corresponding to the brain fatigue level, the mood level, and the stress level of the subject belongs in the three-dimensional space.

Examples of improvement plans are shown in Table 2.

TABLE 2

| | | | | | | Coping |
|---|---|---|---|---|---|---|
| | | | | | | Cognitive |
| | Exercise | | | Meditation | | behavioral |
| Type | Jogging | Stretching | Trekking | Mindfulness | Yoga | therapy |
| A | ◯ | ◯ | ◯ | ◯ | ◯ | |
| B | | ◯ | | | ◯ | ◯ |
| C | | | | | | ◯ |
| D | | | | | | |
| . . . | | | | | | |

For example, when a given subject belongs to the per-type category A, the emotion expression engine section 111 can notify jogging, stretching, trekking, mindfulness, and yoga marked by circles as improvement plans. In addition, when another subject belongs to the per-type category B, the emotion expression engine section 111 can notify stretching, yoga, and cognitive behavioral therapy marked by circles as improvement plans. In this manner, the emotion expression engine section 111 can propose suitable improvement plans in accordance with a per-type category to which a subject belongs.

As described above, we provide an information processing system and the like which are capable of acquiring not only quantitative data such as a pulse wave/heart rate acquired from a pulse wave meter but also data of a stress check result and data such as voice, a facial expression image or the like of the subject during counseling using a video call, calculating values of a stress level, a brain fatigue level, and a mood level from the pieces of data and, by plotting the calculated values in a three-dimensional space defined by an X-axis, a Y-axis, and a Z-axis, visualizing the subject's emotion and the like and enabling diagnosis and treatment to be assisted, thereby realizing analysis of emotion of the subject with high accuracy, realizing early detection of mental illness, and contributing towards solving social problems.

### INDUSTRIAL APPLICABILITY

The information processing system and the like are applicable to a wide range of applications including stress checks at businesses, by individuals, at educational establishments and the like, mental training in sports, improving concentration during learning, measuring mentality during employment examinations and the like, for example.

1-31. (canceled)

32. An information processing device connected to a terminal device of a subject and which visualizes emotion of the subject, the information processing device comprising:
a data managing section that acquires at least data related to voice, a facial expression image, and a pulse wave of the subject;
an emotion expression engine section which calculates a brain fatigue level based on a frequency of the voice, which calculates a mood level by extracting an emotion of the subject from the facial expression image, and which calculates a stress level by performing a frequency analysis of the pulse wave by fast Fourier transform and extracting a high-frequency section and a low-frequency section; and
a three-axes processing section which displays a graph of points plotted at coordinates corresponding to the brain fatigue level, the mood level, and the stress level in a three-dimensional space defined by an X-axis, a Y-axis, and a Z-axis, wherein
the data related to the voice is acquired by making an audio recording of at least a part of a video call with the subject via the terminal device,
the data related to the facial expression image is acquired by making a video recording of at least a part of a video call with the subject via the terminal device, and
the data related to the pulse wave is acquired via the terminal device from a pulse wave meter that measures a pulse wave of the subject.

33. The information processing device according to claim 32, wherein
the data managing section associates the data related to the voice, the facial expression image, and the pulse wave of the subject with dates and times of acquisition of the data and stores the data in storage means of the information processing device, and
the three-axes processing section displays a graph of points plotted according to a time series at coordinates corresponding to the brain fatigue level, the mood level, and the stress level of the subject for each of the dates and times in the three-dimensional space.

34. The information processing device according to claim 32, wherein
the three-dimensional space is divided into a plurality of per-type classification categories, and
the three-axes processing section notifies a category to which a point of coordinates corresponding to the brain fatigue level, the mood level, and the stress level of the subject belongs among the plurality of per-type classification categories in the three-dimensional space.

35. The information processing device according to claim 34, wherein
an improvement plan to be proposed to the subject is determined for each of the plurality of per-type categories, and
the emotion expression engine section notifies the improvement plan with respect to the category to which a point of coordinates corresponding to the brain fatigue level, the mood level, and the stress level of the subject belongs in the three-dimensional space.

36. The information processing device according to claim 32, wherein
the data related to the voice is data acquired by making a continuous audio recording of the voice of the subject reading out loud predetermined fixed phrases displayed on the terminal device at least until a predetermined audio recording time is reached during a video call with the subject via the terminal device.

37. The information processing device according to claim 36, wherein
the emotion expression engine section executes a cerebral activity index measurement algorithm for measuring CEM values that each represents a cerebral activity index to acquire one or more of the CEM values for each subject from the data related to the voice, and
the brain fatigue level is an average value of the one or more CEM values.

38. The information processing device according to claim 32, wherein
the data related to the pulse wave is data acquired by dividing a pulse wave measured by the pulse wave meter into sections, each section being a predetermined time interval.

39. The information processing device according to claim 38, wherein
the emotion expression engine section divides, for each section of the pulse wave, the pulse wave in the section into Hamming windows and calculates, with respect to the pulse wave in each of the Hamming windows, a pulse interval PPI being an interval from a peak to a next peak of the pulse wave of one heartbeat and a time of day,
the emotion expression engine section generates, for each section of the pulse wave, a time-PPI graph which plots a point at coordinates corresponding to the pulse interval PPI and the time of day in a two-dimensional space defined by time of day as an axis of abscissa and PPI as an axis of ordinate,
the emotion expression engine section interpolates between discrete values in the time domain-PPI graph and applies a fast Fourier transform FFT, and calculates an LF value corresponding to the low-frequency component, an HF value corresponding to the high-frequency component, and an LF/HF value by respectively integrating a power spectral density PSD of a result of the FFT in the low-frequency section and in the high-frequency section, and
the stress level is based on at least one value among the LF value, the HF value, and the LF/HF value.

40. The information processing device according to claim 32, wherein
the low-frequency section is 0.04 Hz or higher and lower than 0.15 Hz, and
the high-frequency section is 0.15 Hz or higher and lower than 0.4 Hz.

41. The information processing device according to claim 32, wherein
the data related to the facial expression image is data acquired by making a continuous video recording of a moving image of a facial expression of the subject until at least a predetermined video recording time is reached during a video call with the subject via the terminal device.

42. An information processing method executed in a server connectable to a terminal device of a subject via a network, the information processing method comprising the steps of:
acquiring at least data related to voice, a facial expression image, and a pulse wave of the subject from the terminal device;
calculating a brain fatigue level based on a frequency of the voice, calculating a mood level by extracting an emotion of the subject from the facial expression image, and calculating a stress level by performing a frequency analysis of the pulse wave by fast Fourier transform and extracting a high-frequency section and a low-frequency section; and
displaying a graph of points plotted at coordinates corresponding to the brain fatigue level, the mood level, and the stress level in a three-dimensional space defined by an X-axis, a Y-axis, and a Z-axis, wherein
the data related to the voice is acquired by making an audio recording of at least a part of a video call with the subject via the terminal device,
the data related to the facial expression image is acquired by making a video recording of at least a part of a video call with the subject via the terminal device, and
the data related to the pulse wave is acquired via the terminal device from a pulse wave meter that measures a pulse wave of the subject.

43. The information processing method according to claim 42, wherein
the step of acquiring data related to voice, a facial expression image, and a pulse wave of the subject includes a step of associating the data related to the voice, the facial expression image, and the pulse wave of the subject with dates and times of acquisition of the data and storing the data in storage means of the server, and
the step of displaying the graph includes a step of displaying a graph of points plotted according to a time series at coordinates corresponding to the brain fatigue level, the mood level, and the stress level of the subject for each of the dates and times in the three-dimensional space.

44. The information processing method according to claim 42, wherein
the three-dimensional space is divided into a plurality of per-type classification categories, and
the step of displaying the graph includes a step of notifying a category to which a point of coordinates corresponding to the brain fatigue level, the mood level, and the stress level of the subject belongs among the plurality of per-type classification categories in the three-dimensional space.

45. The information processing method according to claim 44, wherein
an improvement plan to be proposed to the subject is determined for each of the plurality of per-type categories, and
the step of calculating the brain fatigue level, the mood level, and the stress level includes a step of notifying the improvement plan with respect to the category to which a point of coordinates corresponding to the brain fatigue level, the mood level, and the stress level of the subject belongs in the three-dimensional space.

46. The information processing method according to claim 42, wherein
the data related to the voice is data acquired by making a continuous audio recording of the voice of the subject reading out loud predetermined fixed phrases displayed on the terminal device at least until a predetermined audio recording time is reached during a video call with the subject via the terminal device.

47. The information processing method according to claim 46, wherein
the step of calculating the brain fatigue level, the mood level, and the stress level includes a step of executing a cerebral activity index measurement algorithm for measuring CEM values that each represents a cerebral activity index to acquire one or more of the CEM values for each subject from the data related to the voice, and
the brain fatigue level is an average value of the one or more CEM values.

48. The information processing method according to claim 42, wherein
the data related to the pulse wave is data acquired by dividing a pulse wave measured by the pulse wave meter into sections, each section being a predetermined time interval.

49. The information processing method according to claim 48, wherein
the step of calculating the brain fatigue level, the mood level, and the stress level includes:
a step of dividing, for each section of the pulse wave, the pulse wave in the section into Hamming windows and calculating, with respect to the pulse wave in each of the Hamming windows, a pulse interval PPI being an interval from a peak to a next peak of the pulse wave of one heartbeat and a time of day;
a step of generating, for each section of the pulse wave, a time-PPI graph which plots a point at coordinates corresponding to the pulse interval PPI and the time of day in a two-dimensional space defined by time of day as an axis of abscissa and PPI as an axis of ordinate; and
a step of interpolating between discrete values in the time domain-PPI graph and applying a fast Fourier transform FFT, and calculating an LF value corresponding to the low-frequency component, an HF value corresponding to the high-frequency component, and an LF/HF value by respectively integrating a power spectral density PSD of a result of the FFT in the low-frequency section and in the high-frequency section, and
the stress level is based on at least one value among the LF value, the HF value, and the LF/HF value.

50. The information processing method according to claim 42, wherein
the low-frequency section is 0.04 Hz or higher and lower than 0.15 Hz, and
the high-frequency section is 0.15 Hz or higher and lower than 0.4 Hz.

51. An information processing system, comprising:
the information processing device according to claim 32; and
a terminal device capable of accessing the information processing device via a network, wherein
the terminal device transmits at least the data related to the voice, the data related to the facial expression image, and the data related to the pulse wave to the information processing device, and
the information processing device receives the data related to the voice, the data related to the facial expression image, and the data related to the pulse wave, transmits the brain fatigue level, the mood level, and the stress level calculated based on the respective pieces of received data to the terminal device, and displays, on the terminal device, a graph of points plotted at coordinates corresponding to the brain fatigue level, the mood level, and the stress level in a three-dimensional space defined by an X-axis, a Y-axis, and a Z-axis.
